(19) United States
(12) Patent Application Publication
(10) Pub. No.: US 2026/0273150 A1
(43) Pub. Date: Sep. 17, 2026

(54) CONTINUOUS AMBULATORY PERITONEAL DIALYSIS DEVICE BY MANUAL OPERATION

(71) Applicant: GAMBRO LUNDIA AB, Lund (SE)

(72) Inventors: Narendar Venkatachalapathy, Bangalore (IN); Sandesh Subray, Bangalore (IN); Raghavendra HN, Bengaluru (IN); Abhisheck Chodagiri, Banaglore (IN)

(21) Appl. No.: 19/563,396

(22) Filed: Mar. 11, 2026

(30) Foreign Application Priority Data
Mar. 12, 2025 (IN) 202541022315

(51) Int. Cl. A61M 1/28

(52) U.S. Cl. CPC A61M 1/284

(57) ABSTRACT

Apparatus, systems, and methods for an innovative manual device for CAPD therapy are provided. A CAPD device includes a top housing and a base housing configured to surround a Y-junction of a CAPD tubing set. The top housing includes a dial and occlusion surfaces configured to occlude different combinations of tubes of the CAPD tubing set as the dial is turned manually. Markers are provided on a top plate of the top housing to denote to a user to what point in CAPD therapy the dial is set. The markers allow for a CAPD therapy to be conducted sequentially by rotation of the dial.

### CROSS-REFERENCE TO RELATED APPLICATIONS

The present application claims priority to and the benefit of IN Provisional Patent Application No. 202541022315, filed Mar. 12, 2025, entitled “INNOVATIVE CAPD DEVICE BY MANUAL OPERATION”, the disclosures of which are hereby incorporated by reference in their entirety.

### BACKGROUND

Peritoneal dialysis (PD) is a method for treating patients suffering from renal failure. During PD, the peritoneal cavity of a patient is filled with fresh PD fluid, and waste and fluid is transported from the blood of the patient, via the patient's peritoneal membrane, to the fresh PD fluid. The used PD fluid (also referred to as “effluent”) is thereafter drained from the patient. There are several kinds of PD. In automated peritoneal dialysis (APD) a machine or cycler is used to fill the peritoneal cavity with fresh PD fluid, and after a specified dwell period, automatically drains the used PD solution from the body. This procedure is repeated several times, typically during overnight. However, in continuous ambulatory peritoneal dialysis (CAPD), a patient or caregiver performs the dialysis manually by connecting a dialysis fluid bag and a drain bag to a catheter in the patient's abdomen and following a series of steps to drain effluent from the patient, flush the fluid lines, and fill the patient's peritoneum with dialysis fluid. The above process is typically repeated three or four times each day as necessary. Working adults may perform an exchange at home before leaving for work, one at work during their lunch hour, one when the patient arrives home from work and one just before the patient goes to bed.

The CAPD process can be mentally taxing to memorize, as it comprises a long sequence of steps including: (i) preparing the supplies; (ii) putting on a mask and washing hands; (iii) removing a yellow pull ring from a dialysis fluid/drain twin bag set; (iv) removing a minicap from a patient transfer set; (v) connecting the twin bag set to the patient transfer set; (vi) clamping a fill line of the patient transfer set with a clamp; (vii) breaking a frangible of a dialysis fluid bag; (viii) hanging the dialysis fluid bag and placing a drain bag in a basin or on a floor; (ix) opening the patient transfer set to a drain configuration; (x) observing effluent for cloudiness; (xi) closing the patient transfer set; (xii) removing the clamp from the fill line; (xiii) counting to five while new dialysis fluid flows into the drain bag (known as flushing); (xiv) clamping the drain line with a clamp; (xv) opening the patient transfer set to fill; (xvi) when filling is complete, closing the patient transfer set; (xvii) clamping the fill line with a second clamp; (xviii) putting on a mask and washing hands; (xix) disconnecting the twin bag set from the transfer set; and (xx) placing the minicap on the patient transfer set. As may be appreciated from the above sequence, most of these steps involve a physical action to be performed by the user, and these actions are usually performed by opening or closing separate clamps provided with the patient transfer set and/or twin bag set. However, certain populations find it difficult to remember this lengthy sequence and to perform the necessary acts therein due to constraints on dexterity. Finally, CAPD is typically a manual process, not relying on electrical connections that require extra batteries or wall connections.

A need accordingly exists for an apparatus that reduces the amount of steps necessary to be memorized by a CAPD patient or caregiver. Any simplification of the process could work to reduces the reliance of patients on caregivers and reduce the mental burden associated with CAPD. Further, a need exists for an apparatus that facilitates dexterity in the CAPD process. Finally, a need exists for the above apparatus to be capable of manual operation (e.g. not including any electrical components).

### SUMMARY

Example systems, methods, and apparatus are disclosed herein for reducing the amount of steps a patient must memorize to perform CAPD. Specifically, systems, methods, and apparatus for a manually operated and simplified CAPD device is provided.

In light of the disclosure herein, which may be combined with any other aspect listed herein unless specified otherwise, a manual CAPD device comprises a top housing, the top housing comprising a dial, an occluder plate including an extrusion, the extrusion including a groove configured to accept a tongue of the dial, thereby coupling the dial to the occluder plate, and a plurality of occlusion surfaces. The top housing further comprises a top plate including a central aperture, the central aperture configured to at least partially surround the extrusion of the occluder plate, and a top housing hinge. The device further comprises a base housing, the base housing comprising a plurality of tubing pins configured to engage at least one tubing line, a base housing hinge configured to align with the top housing hinge when the device is in a closed configuration, and a hinge pin configured to rotatably couple the top housing hinge and the base housing hinge, wherein the plurality of occlusion surfaces is configured to occlude the at least one tubing line, and wherein rotation of the dial about a central axis of the device causes the occluder plate to rotate.

In another aspect of the present disclosure, which may be combined with any other aspect listed herein unless specified otherwise, the at least one tubing line comprises a patient line, a drain line, and a dialysis fluid line.

In another aspect of the present disclosure, which may be combined with any other aspect listed herein unless specified otherwise, the top plate further comprises a plurality of markers, wherein each of the plurality of markers denotes a different therapy configuration when the dial is rotated to align with that marker.

In another aspect of the present disclosure, which may be combined with any other aspect listed herein unless specified otherwise, the plurality of markers comprises five markers.

In another aspect of the present disclosure, which may be combined with any other aspect listed herein unless specified otherwise, a first marker corresponds to a first closed configuration, the second marker corresponds to a drain configuration, the third marker corresponds to a flush configuration, the fourth marker corresponds to a fill configuration, and the fifth marker corresponds to ta second closed configuration.

In another aspect of the present disclosure, which may be combined with any other aspect listed herein unless specified otherwise, the plurality of occlusion surfaces comprises a first occlusion surface, a second occlusion surface, and a third occlusion surface, wherein each of the plurality of occlusion surfaces is disposed radially about the central axis, and wherein each of the plurality of occlusion surfaces forms a partial circumference.

In another aspect of the present disclosure, which may be combined with any other aspect listed herein unless specified otherwise, each of the plurality of occlusion surfaces is disposed at a constant distance from the central axis relative to each other occlusion surface.

In another aspect of the present disclosure, which may be combined with any other aspect listed herein unless specified otherwise, when the dial is rotated to align with the first marker, the first occlusion surface is configured to occlude the drain line and the dialysis fluid line, the third occlusion surface is configured to occlude the patient line, and the second occlusion surface is configured to not occlude any tubing line.

In another aspect of the present disclosure, which may be combined with any other aspect listed herein unless specified otherwise, when the dial is rotated to align with the second marker, the first occlusion surface is configured to occlude the dialysis fluid line, and the second occlusion surface and the third occlusion surface are configured to not occlude any tubing line.

In another aspect of the present disclosure, which may be combined with any other aspect listed herein unless specified otherwise, when the dial is rotated to align with the third marker, the second occlusion surface is configured to occlude the patient line, and the first occlusion surface and the third occlusion surface are configured to not occlude any tubing line.

In another aspect of the present disclosure, which may be combined with any other aspect listed herein unless specified otherwise, when the dial is rotated to align with the fourth marker, the third occlusion surface is configured to occlude the drain line, and the first occlusion surface and the second occlusion surface are configured to not occlude any tubing line.

In another aspect of the present disclosure, which may be combined with any other aspect listed herein unless specified otherwise, when the dial is rotated to align with the fifth marker, the first occlusion surface is configured to occlude the patient line, the third occlusion surface is configured to occlude the drain line and the dialysis fluid line, and the second occlusion surface is configured to not occlude any tubing line.

In another aspect of the present disclosure, which may be combined with any other aspect listed herein unless specified otherwise, an angle between the drain line and the dialysis fluid line is smaller than either of an angle between the drain line and the patient line and an angle between the dialysis fluid line and the patient line when the at least one tubing line is engaged with the plurality of tubing pins.

In another aspect of the present disclosure, which may be combined with any other aspect listed herein unless specified otherwise, an angle between the drain line and the dialysis fluid line, an angle between the drain line and the patient line, and an angle between the dialysis fluid line and the patient line are all substantially equal when the at least one tubing line is engaged with the plurality of tubing pins.

In another aspect of the present disclosure, which may be combined with any other aspect listed herein unless specified otherwise, the dial further comprises a dial aperture configured to allow each of the plurality of markers to be viewed through the dial aperture when the dial is rotated to align with each of the plurality of markers.

In another aspect of the present disclosure, which may be combined with any other aspect listed herein unless specified otherwise, the base housing further comprises a locking pin configured to extend into a locking channel of the occluder plate when the device is in a closed configuration.

In another aspect of the present disclosure, which may be combined with any other aspect listed herein unless specified otherwise, the locking pin comprises an enlargement.

In another aspect of the present disclosure, which may be combined with any other aspect listed herein unless specified otherwise, the locking channel comprises at least one end aperture, wherein a diameter of the at least one end aperture is larger than a diameter of the enlargement.

In another aspect of the present disclosure, which may be combined with any other aspect listed herein unless specified otherwise, the locking channel comprises at least one lip configured to interfere with a bottom surface of the enlargement in at least one rotational position of the locking channel.

In another aspect of the present disclosure, which may be combined with any other aspect listed herein unless specified otherwise, the device further comprises a latch configured to align with a base latch plate of the base housing and a top latch plate of the top housing, wherein the latch comprises a latch through hole, wherein the base latch plate comprises a base latch plate aperture, wherein the top latch plate comprises a top latch plate aperture, and wherein each of the latch through hole, the base latch plate aperture, and the top latch plate aperture are configured to align and accept a latch pin such that the latch couples the top housing to the base housing in a closed configuration of the device.

In light of the present disclosure and the above aspects, it is therefore an advantage of the present disclosure to provide a device which automates the CAPD process by implementing part of the CAPD sequence by turning a dial to a plurality of positions.

It is another advantage of the present disclosure to provide a manual apparatus which reduces the amount of dexterity necessary to perform the CAPD sequence.

It is another advantage of the present disclosure to provide a reusable device which may be coupled to existing transfer sets and twin bag sets, and which does not require constant sterilization.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. In addition, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1A shows an isometric front view of a CAPD device, according to an example embodiment of the present disclosure.

FIG. 1B shows a top view of the CAPD device of FIG. 1A.

FIG. 2 shows an isometric exploded view of the CAPD device of FIG. 1A.

FIG. 3A shows an isometric view of the CAPD device of FIG. 1A in an open configuration.

FIG. 3B shows a front view of the CAPD device of FIG. 3A.

FIG. 4A shows an isometric view of a base housing of the CAPD device of FIG. 1A.

FIG. 4B shows a top view of the base housing of FIG. 4A with CAPD tubing installed.

FIG. 5 shows an isometric exploded view of a top housing of the CAPD device of FIG. 1A.

FIG. 6A shows an isometric view of the top housing of FIG. 5 in a first configuration.

FIG. 6B shows a bottom view of the top housing of FIG. 6A.

FIG. 7A shows an isometric view of the top housing of FIG. 5 in a second configuration.

FIG. 7B shows a bottom view of the top housing of FIG. 7A.

FIG. 8A shows an isometric view of the top housing of FIG. 5 in a third configuration.

FIG. 8B shows a bottom view of the top housing of FIG. 8A.

FIG. 9A shows an isometric view of the top housing of FIG. 5 in a fourth configuration.

FIG. 9B shows a bottom view of the top housing of FIG. 9A.

FIG. 10A shows an isometric view of the top housing of FIG. 5 in a fifth configuration.

FIG. 10B shows a bottom view of the top housing of FIG. 10A.

FIGS. 11A-11B illustrate configurations of test setups for validating use of the CAPD device.

FIGS. 12A-12B illustrate graphical versions of test data for validating use of the CAPD device in different configurations.

### DETAILED DESCRIPTION

Methods, systems, and apparatus are disclosed herein for reducing the amount of steps a patient must memorize to perform CAPD. Specifically, systems, methods, and apparatus for a manually operated and simplified CAPD device are provided.

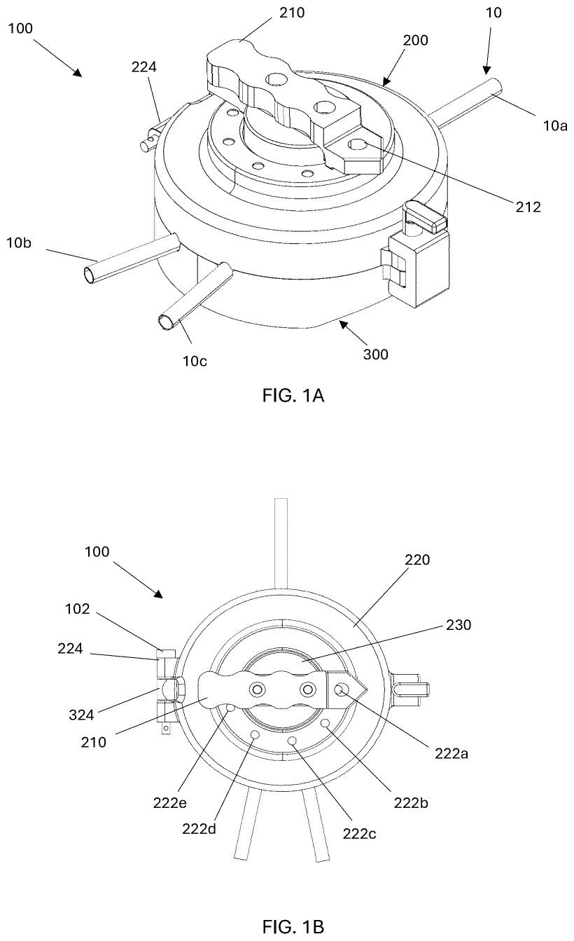

Turning now to FIGS. 1A-1B, a CAPD device 100 is configured to surround the tubing 10 of a CAPD system, specifically at the junction between the patient line 10a and the drain line 10b and dialysis fluid line 10c of a twin bag set. This junction may be referred to as a “Y-junction” because of the “Y” shape created at the interface of the patient line 10a to the twin bag set lines 10b, 10c. The CAPD device 100 includes a top housing 200 and a base housing 300 hingedly coupled by a top housing hinge 224 and a base housing hinge 324 with a hinge pin 102 set therebetween.

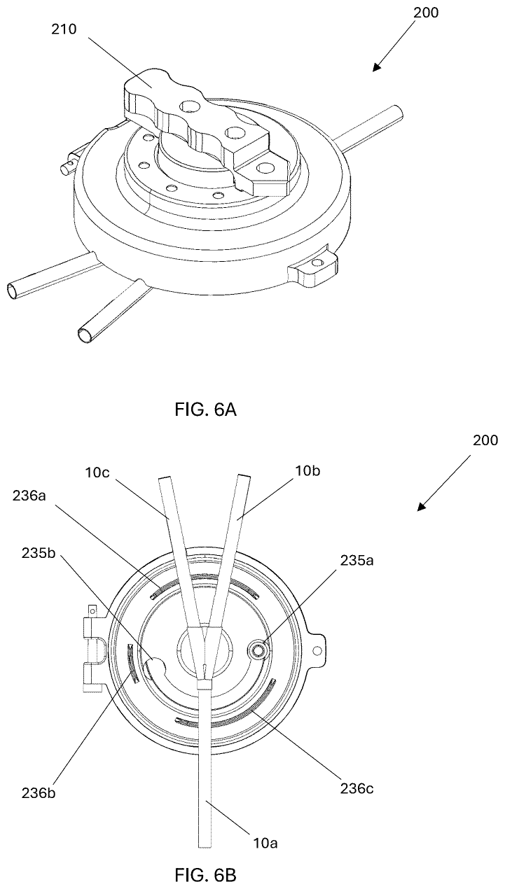

The top housing 200 includes a dial 210, a top plate 220, and an occluder plate 230. The top housing hinge 224 is provided on the top plate 220, such that the top plate 220 is stationary relative to the base housing 300 while the hinge pin 102 is in place. The top plate 220 features a plurality of markers 222a-e that are spaced at an angle from each adjacent marker 222a-e. In a preferred embodiment, each marker 222a-e is disposed at a 40 degree angle from each adjacent marker 222a-e. Although five markers 222a-e are illustrated, any number of markers may be used to denote a step in the CAPD sequence according to the expediencies of a given CAPD sequence. A dial aperture 212 disposed on a first end of the dial allows a user to see which marker 222a-e the dial 210 is currently disposed toward (and therefore at which step in the CAPD process the CAPD device 100 is currently set, as will be appreciated from the description of FIGS. 6A-10B). Although not illustrated, each marker 222a-e may have an associated marking or sticker indicating the CAPD therapy step associated with that marker 222a-e. For example, the first marker 222a may have a marking that is labeled “1” or “closed”, the second marker 222b may have a label that reads “2” or “drain”, the third marker 222c may have a label that reads “3” or “flush”, the fourth marker 222d may have a label that reads “4” or “fill”, and the fifth marker 222e may have a label that reads “5” or “closed”. The label may be within the marker 222a-e or disposed on a portion of the top plate 220 adjacent to each marker 222a-e. Other labels or markings, such as arrows and/or high contrast colors, may be utilized to further distinguish which position the dial 210 is set to currently. The dial 210 optionally comes to a point on an end closest to the dial aperture 212 so that a patient can more easily distinguish which position the dial 210 is currently in.

The dial 210 is rigidly coupled to the occluder plate 230, such that as the dial 210 rotates about a central axis 110, the occluder plate 230 rotates with the dial 210. The dial 210 in some embodiments has curved surfaces to enhance the ergonomics of the dial 210 and reduce the threshold dexterity required to actuate (rotate) the dial 210. In some embodiments, the markers 222a-e include a depression into the top plate 220 and the dial aperture 212 includes a spring-loaded ball bearing or pin that sinks into the markers 222a-e as the dial is twisted to create haptic feedback for a user when the dial 210 has reached each marker's 222a-e position.

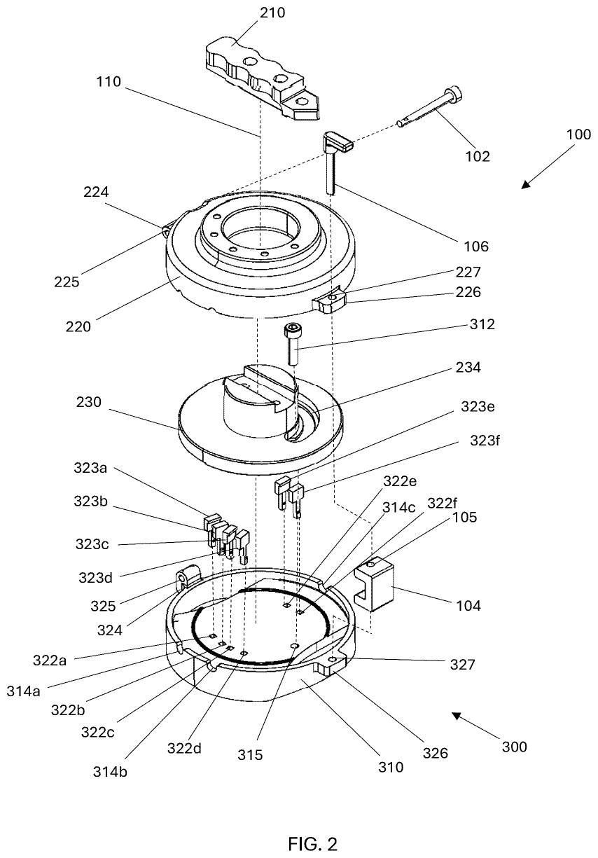

FIG. 2 illustrates an exploded view of the CAPD device 100 in an illustrative configuration without the tubing 10 installed for the sake of clarity. The top housing hinge 224 includes a top housing hinge aperture 225 which aligns with a base housing hinge aperture 325 of the base housing hinge 324, allowing the hinge pin 102 to be placed therebetween and secure a hinged connection between the top housing 200 and the base housing 300. A top latch plate 226 with top latch plate aperture 227 is also provided on the top plate 220 and which corresponds to a base latch plate 326 with base latch plate aperture 327 provided on the base plate 310. The top latch plate aperture 227 is configured to align with the base latch plate aperture 327 as well as a latch through hole 105 of a latch 104 when the top housing 200 is hingedly coupled to the base housing 300. A latch pin 106 is then configured to be inserted through each of the latch through hole 105, the top latch plate aperture 227, and the base latch plate aperture to secure the top housing 200 to the base housing 300 after the tubing 10 is installed and the top housing 200 is drawn to the base housing 300 about the hinge pin 102.

The tubing 10 is held resiliently coupled to the base plate 310 by a plurality of tubing pins 323a-f. The tubing pins 323a-f are configured to be coupled to the base plate 310 by a plurality of tubing pin channels 322a-f. In an alternative embodiment, the tubing pins 323a-f are formed as an integrated feature of the base plate 310 or otherwise permanently affixed into the tubing pin channels 322a-f. It may be preferable to employ tubing pin channels 322a-f in order that the tubing pins 323a-f may more easily be replaced in case of failure. The tubing pins 323a-f are generally divided into three pairs, each pair configured to resiliently hold one line 10a-c of the tubing 10 by a lip disposed toward the other tubing pin 323a-f in the pair. In a preferred embodiment, the tubing 10 is press fit between a corresponding pair of tubing pins 323a-f. In an alternative embodiment, the tubing 10 is laid across the base plate 310 and then the tubing pins 323a-f are installed into the tubing pin channels 322a-f over the tubing 10 to hold the tubing 10 in place. In an alternative embodiment each pair of tubing pins 323a-f is replaced by a single gantry or arch which surrounds the tubing 10 to hold it in place. Tubing channels 314a-c are provided on the side face of the base plate 310 so that the tubing 10 is not occluded when the top housing 200 is closed toward the base housing 300 and latched by the latch 104 and latching pin 106. In a preferred embodiment, the tubing channels 314a-c extend into the top plate 220 as well.

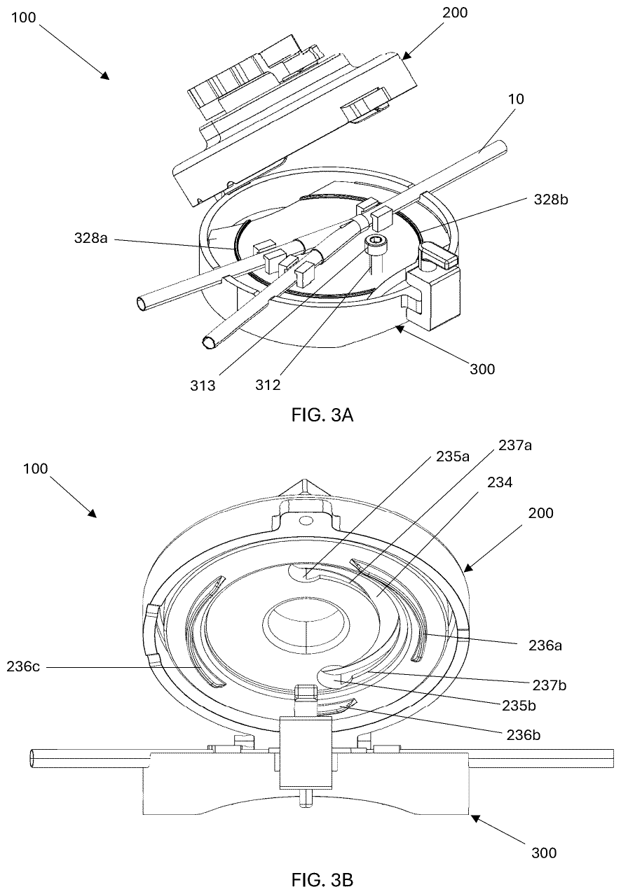

A locking pin 312 is further provided and coupled to the base plate 310 by a locking pin channel 315. In an alternative embodiment, the locking pin 312 is formed as an integrated feature of the base plate 310 or otherwise permanently affixed into the locking pin channel 315. As will be described in further detail in the description of FIGS. 3A-3B, the locking pin 312 sits along the locking channel 234 of the occluder plate 230 and ensures that a patient or user is not able to set the CAPD device 100 to the open configuration while the dial 210 is set to a position that is not the start or the end of the therapy sequence. If the CAPD device 100 is set to the open position during one of the intermediate steps of the sequence, this could lead to effluent entering the fill line or prematurely entering the drain line, or it could cause effluent re-entering the peritoneum of the patient, amongst other issues which could result in infection, waste of dialysis fluid, etc.

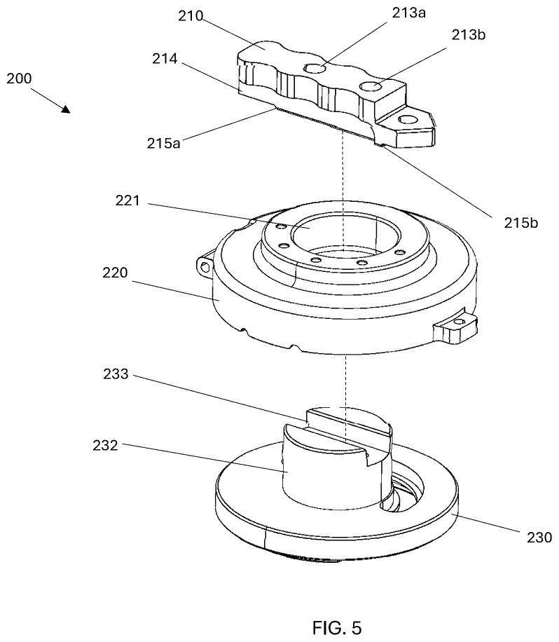

The dial 210 is rigidly coupled to the occluder plate 230, and the top plate 220 is partially inserted therebetween, as will be described in further detail in the description of FIG. 5. The locking pin 312 thereby also ensures that the dial 210 cannot be rotated outside of the positions necessary to perform a CAPD therapy while the CAPD device 100 is in a closed configuration. In an alternative embodiment, a ratchet is provided on the dial 210, which does not allow the dial 210 to move to a previous marker 222a-e until the dial 210 reaches the fifth marker 222e. This ensures that the CAPD therapy is run to completion without interference.

Turning now to FIGS. 3A-3B, the CAPD device 100 is shown in an open configuration, wherein the top housing 200 is drawn away from the base housing 300 about the hinged coupling in order to load a set of tubing 10. The locking pin 312 includes an enlargement 313 which is sized to fit within end apertures 235a,b of the locking channel 234. Enlargement 313 also includes a diameter that is larger than any portion of the locking channel 234 formed by the lips 237a,b of the locking channel 234 between the end apertures 235a,b. In this way, the locking pin 312 can fit into the top housing 200 at the beginning of therapy (when the first end aperture 235a is disposed over the locking pin) and at the end of therapy (when the second end aperture 235b is disposed over the locking pin), but will find interference with the walls of the locking channel 234 at any other point in therapy. This ensures that the CAPD device 100 will not be set to the open configuration in the middle of a therapy.

The base housing 300 further includes force concentration surfaces 328a,b which interface with occlusion surfaces 236a-c of the top housing in order to occlude the tubing 10 in different configurations (as will be discussed in further detail in the descriptions of FIGS. 6A-10B). The occlusion surfaces 236a-c may each have rounded or tapered ends in order to create a surface which more easily slides over the tubing 10 (i.e. reduces the force necessary to create an occlusion in the tubing 10 when the dial 210 is being twisted). In a preferred embodiment, the occlusion surfaces 236a-c extend in a circular configuration about the central axis 110 at a constant radius, each forming a partial circumference. In alternative embodiments, at least two of the occlusion surfaces 236a-c are disposed at a different radial distance to the central axis 110. In a preferred embodiment, the first occlusion surface 236a and the third occlusion surface 236c have a greater length about the partial circumference formed than the partial circumference formed by the second occlusion surface 236b, as may be appreciated from FIG. 3B.

As shown in FIGS. 3A-3B, the hinged coupling between the top housing 200 and the base housing 300 allows a patient or user to insert the Y-junction of a twin bag set. Once the existing twin bag set Y-junction is set in the base housing 300, the user is able to pivot the top housing 200 toward the base housing 300 to fully surround the Y-junction. Such an arrangement allows for existing twin bag sets to be used with the CAPD device 100 without manipulating the twin bag set or separately attaching any of the lines 10a-c. This further aids in maintaining sterility of the lines 10-c and the fluids therein, since separately attaching the lines 10a-c to connectors on any CAPD device can introduce pathogens into the lines 10a-c. Pathogens can be introduced by touch contamination or from non-sterile air flow. For example, a system which uses force fit connections for each of the lines 10a-c and includes channels within the housing itself for fluid flow to occur has increased opportunities to introduce pathogens into the lines 10a-c or requires additional steps to sterilize the lines 10a-c and the device, which complicates the process and requires additional training for users. Further, because the lines 10a-c are already connected in the twin bag set, there is less risk that the lines 10a-c will be placed in the wrong position compared to systems which utilize separate connections of the lines 10a-c to the device itself. In some embodiments of the present disclosure, the base housing 300 includes depressions.

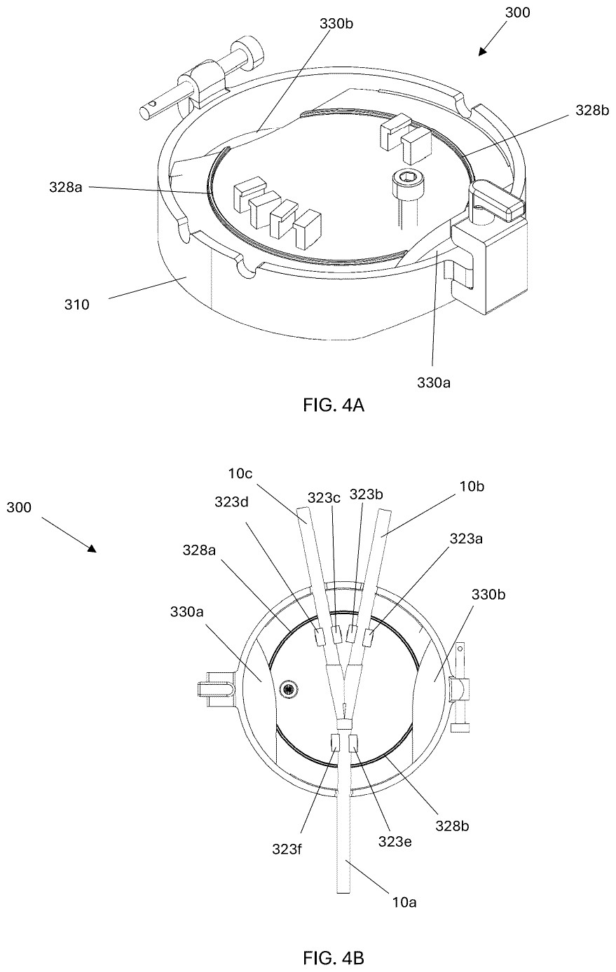

FIGS. 4A-4B show the base housing 300 in greater focus. The force concentration surfaces 328a,b are separated by two channels 330a,b in the base plate 310. In an alternative embodiment, no channels 330a,b are provided in the base plate 310. Instead, there is provided a singular force concentration surface extending circularly on the base plate 310 with no breakages.

The tubing pins 323a-f are configured to hold each of the lines 10a-c at an angle apart from each other. In some embodiments, the drain line 10b and dialysis fluid line 10c are disposed at an angle relative to each other smaller than an angle between either the drain line 10b and the patient line 10a or the dialysis fluid line 10c and the patient line 10a. In other embodiments, each of the lines 10a-c is evenly disposed at a 120 degree angle from each adjacent line 10a-c. The exact angle may be determined by the arrangement of the twin tubing set provided. It will be recognized by those of skill in the art that the positions and lengths of the partial circumferences formed by the occlusion surfaces 236a-c may be adjusted as necessary to accommodate the expediencies of the angles formed by the lines 10a-c so that a CAPD therapy can be sequentially performed according to the description of FIGS. 6A-10B (described further below).

FIG. 5 shows the top housing 200 in greater focus and in an exploded view. The dial 210 includes a tongue 214 configured to mate with a groove 233 on an extrusion 232 of the occluder plate 230. The dial 210 also includes fastener apertures 213a,b which may be used to guide a fastener to the extrusion 232 and rigidly affix the dial 210 to the occluder plate 230. In some embodiments, the fastener apertures 213a,b are countersunk in order to ensure no foreign bodies enter the fastener apertures 213a,b after the dial 210 is affixed to the occluder plate 230. The extrusion 232 is sized to fit through a central aperture 221 of the top plate 220. In this manner, the affixation of the dial 210 to the occluder plate 230 sandwiches the top plate 220 between the dial 210 and the occluder plate 230. Thus the top housing 200 is held together in assembly. The tongue 214 of the dial 210 may include curves or jogs 215a,b configured to create a clearance for the sides of the central aperture 221. The jogs 215a,b allow the dial 210 to overhang the top plate 220 with the smallest clearance possible, which helps prevent debris from entering between the dial 210 and the top plate 220 and also between the top plate 220 and the extrusion 232.

FIGS. 6A-10B illustrate the top housing 200 when the dial 210 is at each of five different configurations, each configuration pertaining to a different part of CAPD therapy. In FIGS. 6A-6B, the dial 210 (and thereby the occluder plate 230) is in a first configuration, corresponding to a first CAPD therapy position, which may for example be a closed position (i.e. where each line 10a-c is occluded). In this configuration, the first occlusion surface 236a is disposed over and occludes the drain line 10b and the dialysis fluid line 10c, while the third occlusion surface 236c is disposed over and occludes the patient line 10a. The second occlusion surface 236b is not occluding any line 10a-c, but each line 10a-c is occluded in the configuration. The first configuration allows the patient to set up the dialysis fluid bag and drain bag, remove any clamps which may have been provided on the bag sets, or otherwise perform necessary acts prior to the beginning of CAPD therapy. In this first configuration the locking pin is disposed at the first end aperture 235a, which means the CAPD device 100 can still be set to the open configuration if necessary (for example, if the placement of the tubing 10 needs to be adjusted before therapy is started).

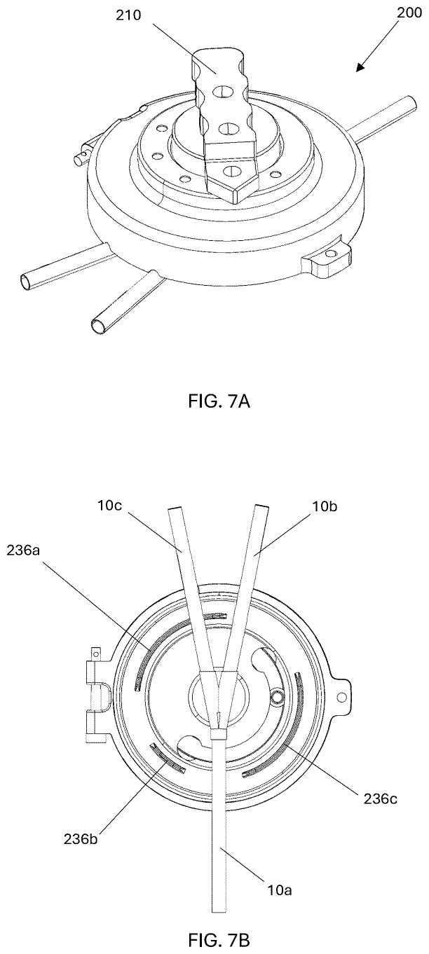

In FIGS. 7A-7B, the dial 210 (and thereby the occluder plate 230) is in a second configuration, corresponding to a second CAPD therapy position, which may for example be a drain position (i.e. where the dialysis fluid line 10c is occluded while the patient line 10a and the drain line 10b are open). In this configuration, the first occlusion surface 236a is disposed over and occludes the dialysis fluid line 10c, while the second occlusion surface 236b and the third occlusion surface 236c are not occluding any line 10a-c. The second configuration allows effluent to flow from the peritoneum of a patient to a drain bag, which drain bag is typically situated on a floor or at least below the level of the patient's insertion line. This drain prepares the peritoneal cavity to receive the dialysis fluid in the following steps.

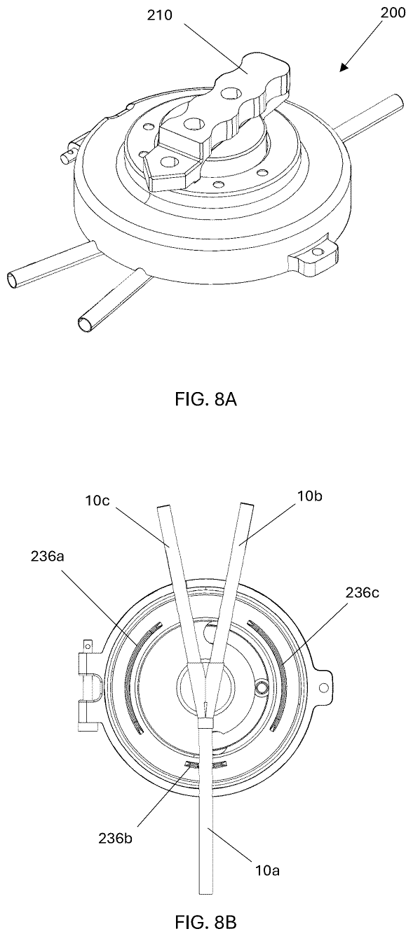

In FIGS. 8A-8B, the dial 210 (and thereby the occluder plate 230) is in a third configuration, corresponding to a third CAPD therapy position, which may for example be a flush position (i.e. where the patient line 10a is occluded while the drain line 10b and the dialysis fluid line 10c are open). In this configuration, the second occlusion surface 236b is disposed over and occludes the patient line 10a, while the first occlusion surface 236a and the third occlusion surface 236c are not occluding any line 10a-c. The third configuration allows dialysis fluid to flow through the dialysis fluid line 10c and empty a portion of the dialysis fluid into the drain line 10b. This is known as a flushing operation, which primes the dialysis fluid line 10c so that no effluent backflows to the patient line 10a during the following fill operation. Some dialysis fluid is lost sacrificially to the drain line 10b to ensure that only fresh dialysis fluid is delivered to the patient during the fill phase.

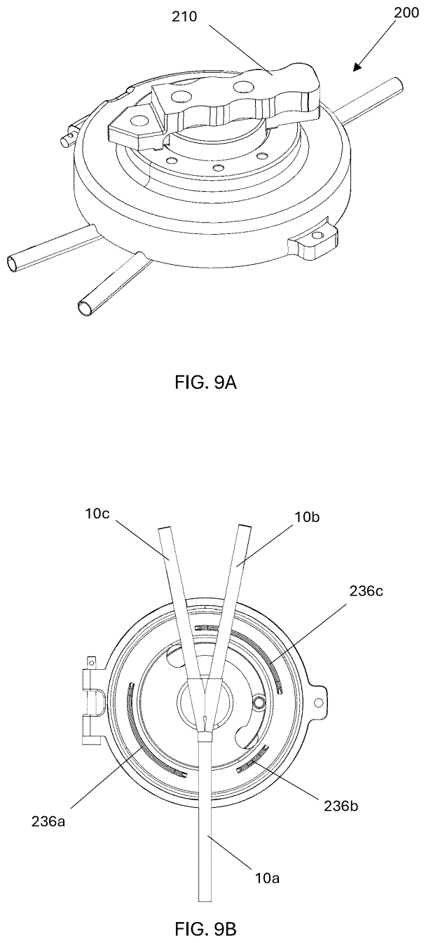

In FIGS. 9A-9B, the dial 210 (and thereby the occluder plate 230) is in a fourth configuration, corresponding to a fourth CAPD therapy position, which may, for example, be a fill position (i.e. where the drain line 10b is occluded while the patient line 10a and the dialysis fluid line 10c are open). In this configuration, the third occlusion surface 236c is disposed over and occludes the drain line 10b, while the first occlusion surface 236a and the second occlusion surface 236b are not occluding any line 10a-c. The fourth configuration allows dialysis fluid to flow through the dialysis fluid line 10c and into the patient line 10a, thereby passing into the peritoneal cavity of a patient. This is known as a fill operation, which delivers the remaining volume of dialysis fluid in the dialysis fluid bag to the peritoneum of the patient. Once the volume of dialysis fluid has been delivered, the dialysis fluid will then be allowed to dwell in the patient's peritoneal cavity, drawing out excess fluid and waste products to become the effluent in the next round of CAPD therapy.

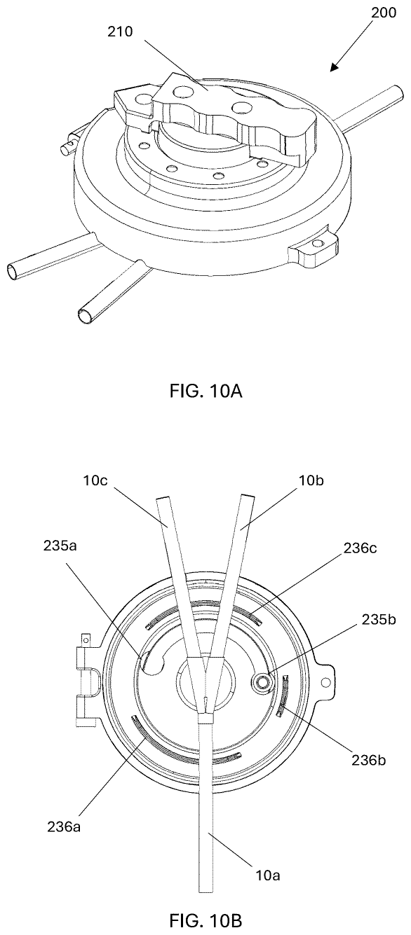

In FIGS. 10A-10B, the dial 210 (and thereby the occluder plate 230) is in a fifth configuration, corresponding to a fifth CAPD therapy position, which may for example be a second closed position (i.e. where each line 10a-c is occluded). In this configuration, the third occlusion surface 236c is disposed over and occludes the drain line 10b and the dialysis fluid line 10c, while the first occlusion surface 236a is disposed over and occludes the patient line 10a. The second occlusion surface 236b is not occluding any line 10a-c. The fifth configuration allows for the twin bag set and the patient transfer set to be prepared for removal now that the dialysis fluid has been delivered to the patient and will be allowed to dwell. In this fifth configuration the locking pin is disposed at the second end aperture 235b, which allows the CAPD device 100 to be set to the open configuration once the twin bag set and the patient transfer set have been prepared for removal. At that point, the user is able to open the CAPD device 100 and remove the lines 10a-c. The CAPD device 100 can then be sterilized if necessary and used for the next CAPD therapy.

### Test Data

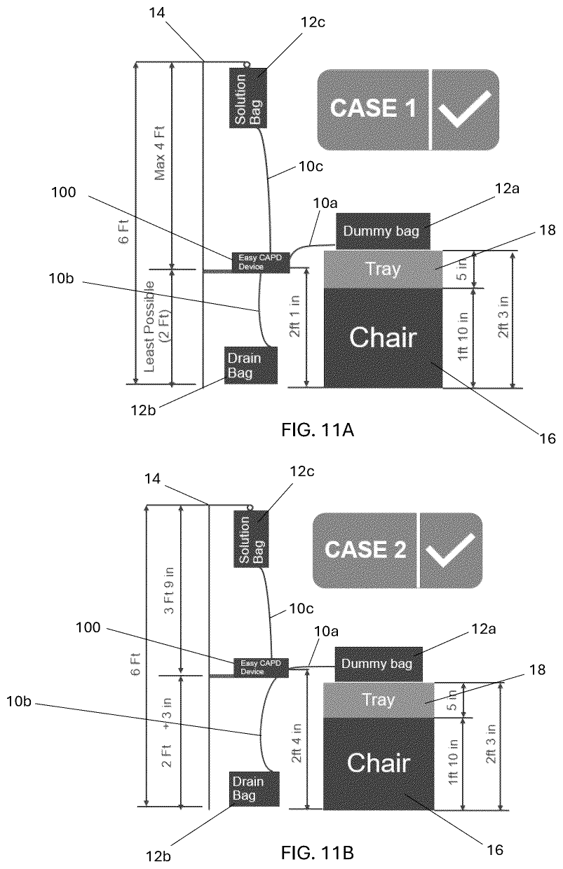

FIGS. 11A-B illustrate two test scenarios conducted using an embodiment of the CAPD device 100 as described herein. CAPD therapy was completed using a dummy bag 12a (connected to patient line 10a) instead of a live patient, with the CAPD device 100 placed at different heights on an IV pole 14. Flow rates were measured during drain and fill cycles in order to validate appropriate flow rates for patient use with the CAPD device 100 connected. An IV pole 14 with a height of six feet was used to place the dialysis fluid solution bag 12c (connected to dialysis fluid line 10c) and the CAPD device 100. The dialysis fluid solution bag 12c was placed at the maximum height of six feet. The drain bag 12b (connected to drain line 10b) was placed on the floor. The placement of the dialysis fluid solution bag 12c and the drain bag 12b were intended to simulate a typical CAPD therapy arrangement. A chair 16 with a height of 22 inches was utilized with a tray 18 having a height of five inches placed thereon, and upon which the dummy bag 12a was placed, creating a total height of 27 inches from the ground for the dummy bag 12a. The height of the CAPD device 100 was then adjusted between the two test configurations. The distance from the CAPD device 100 to the dummy bag 12a was nine inches. Fluid was allowed to completely drain from the dummy bag 12a during the drain cycle and to completely drain from the dialysis fluid solution bag 12c during the fill cycle.

### Test 1

FIG. 11A illustrates a first test configuration in which the CAPD device 100 is placed three inches below the level of the dummy bag 12a, at a height of 24 inches from the ground, which represents the minimum allowable therapeutic height for CAPD therapy. The patient line 10a coming from the CAPD device 100 lies at 25 inches above the ground in this configuration. Three trials were run in this test configuration. The results of these trials are presented in Table 1, below.

TABLE 1

| Trial | Drain Cycle | Drain Flowrate | Fill Cycle | Fill Flowrate |
|---|---|---|---|---|
| No. | Time (min.) | (mL/min.) | Time (min.) | (mL/min.) |
| 1 | 4.22 | 473.9 | 3.4 | 588.2 |
| 2 | 4.01 | 498.8 | 3.25 | 615.4 |
| 3 | 4.06 | 492.6 | 3.24 | 617.3 |

The test results from Table 1 show drain times, drain flowrates, fill times, and fill flowrates which are within acceptable therapeutic limits. As may be appreciated in conjunction with Table 3, described later on, the average drain flowrate (488.4 mL/min.) and average fill flowrate (607.0 mL/min.) are within 7% and 2%, respectively, of the drain flowrate (480.4 mL/min.) and fill flowrate (566.0 mL/min.) without use of the CAPD device 100 under similar conditions. The CAPD device 100 is thus validated as acceptable for use when used at a height of three inches below the patient insert with other normal CAPD therapy conditions observed.

### Test 2

FIG. 11B illustrates a second test configuration in which the CAPD device 100 is placed at the level of the dummy bag 12a, at a height of 27 inches from the ground, which represents a typical suggested therapeutic height for CAPD therapy. The patient line 10a coming from the CAPD device 100 lies at 28 inches above the ground in this configuration. Three trials were run in this test configuration. The results of these trials are presented in Table 2, below.

TABLE 2

| Trial | Drain Cycle | Drain Flowrate | Fill Cycle | Fill Flowrate |
|---|---|---|---|---|
| No. | Time (min.) | (mL/min.) | Time (min.) | (mL/min.) |
| 1 | 4.22 | 473.9 | 3.55 | 563.4 |
| 2 | 4.15 | 481.9 | 3.35 | 597.0 |
| 3 | 4.216 | 474.4 | 3.51 | 568.8 |

The test results from Table 2 show drain times, drain flowrates, fill times, and fill flowrates which are within acceptable therapeutic limits. As may be appreciated in conjunction with Table 3, described later on, the average drain flowrate (476.7 mL/min.) and average fill flowrate (576.4 mL/min.) are within 2% of the drain flowrate (480.4 mL/min.) and fill flowrate (566.0 mL/min.) without use of the CAPD device 100 under similar conditions. The CAPD device 100 is thus validated as acceptable for use when used at a height matching the height of the patient insert with other normal CAPD therapy conditions observed.

### Varying Solution Bag Height

Tests were also performed with the dialysis fluid bag 12c placed at different heights on the IV pole 14 combined with the CAPD device 100 at various heights and with no CAPD device 100. Specifically, the height of the dialysis fluid bag 12c was varied between 48 inches, 60 inches, and 72 inches from the ground. At each height of the dialysis fluid bag 12c, the test was run with no CAPD device 100, a CAPD device 100 at a height of 27 inches from the ground, and a CAPD device at a height of 24 inches from the ground. The results of these trials are presented in Table 3, below.

TABLE 3

| Dialysis Fluid | CAPD Device | Drain Flowrate | Fill Flowrate |
|---|---|---|---|
| Bag Height (in.) | Height (in.) | (mL/min.) | (mL/min.) |
| 48 | No CAPD Device | 496.9 | 320.0 |
| 60 | No CAPD Device | 495.9 | 449.2 |
| 72 | No CAPD Device | 480.4 | 566.0 |
| 48 | 24 | 491.9 | 326.1 |
| 60 | 24 | 495.9 | 471.4 |
| 72 | 24 | 488.4 | 607.0 |
| 48 | 27 | 460.8 | 360.2 |
| 60 | 27 | 485.2 | 478.2 |
| 72 | 27 | 476.7 | 576.4 |

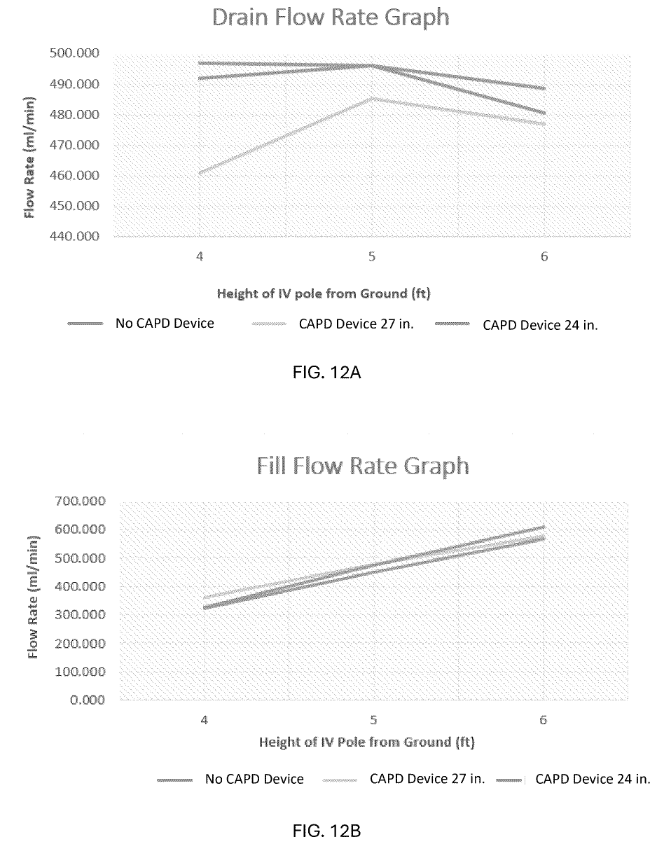

FIGS. 12A and 12B show graphical versions of the data in Table 3. As may be appreciated from Table 3 and the graphs in FIGS. 12A-12B, the greatest deviation between the control sample without the CAPD device and using the CAPD device was a less than 8% deviation in the drain flowrate when the dialysis solution bag 12c was placed at a height of 48 inches and the CAPD device 100 was at a height of 27 inches. All other results were within 3% of the flowrates without a CAPD device 100. Thus, use of the CAPD device 100 is validated as acceptable compared to CAPD therapy without use of a CAPD device 100. It may also be noted that using the CAPD device 100 at a height of 24 inches above the ground may produce results more akin to CAPD therapy without the CAPD device 100.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A CAPD device comprising:
a top housing comprising:
a dial;
an occluder plate including:
an extrusion including a groove configured to accept a tongue of the dial, thereby coupling the dial to the occluder plate; and
a plurality of occlusion surfaces;
a top plate including a central aperture, the central aperture configured to at least partially surround the extrusion of the occluder plate; and
a top housing hinge;
a base housing comprising:
a plurality of tubing pins configured to engage at least one tubing line;
a base housing hinge configured to align with the top housing hinge when the CAPD device is in a closed configuration; and
a hinge pin configured to rotatably couple the top housing hinge and the base housing hinge,
wherein the plurality of occlusion surfaces is configured to occlude the at least one tubing line, and
wherein rotation of the dial about a central axis of the CAPD device causes the occluder plate to rotate.

2. The device of claim 1, wherein the at least one tubing line comprises a patient line, a drain line, and a dialysis fluid line.

3. The device of claim 2, wherein the top plate further comprises a plurality of markers, wherein each of the plurality of markers denotes a different therapy configuration when the dial is rotated to align with that marker.

4. The device of claim 3, wherein the plurality of markers comprises five markers.

5. The device of claim 4, wherein a first marker corresponds to a first closed configuration, a second marker corresponds to a drain configuration, a third marker corresponds to a flush configuration, a fourth marker corresponds to a fill configuration, and a fifth marker corresponds to a second closed configuration.

6. The device of claim 5, wherein the plurality of occlusion surfaces comprises a first occlusion surface, a second occlusion surface, and a third occlusion surface, wherein each of the plurality of occlusion surfaces is disposed radially about the central axis, and wherein each of the plurality of occlusion surfaces forms a partial circumference.

7. The device of claim 6, wherein each of the plurality of occlusion surfaces is disposed at a constant distance from the central axis relative to each other occlusion surface.

8. The device of claim 6, wherein when the dial is rotated to align with the first marker, the first occlusion surface is configured to occlude the drain line and the dialysis fluid line, the third occlusion surface is configured to occlude the patient line, and the second occlusion surface is configured to not occlude any tubing line.

9. The device of claim 6, wherein when the dial is rotated to align with the second marker, the first occlusion surface is configured to occlude the dialysis fluid line, and the second occlusion surface and the third occlusion surface are configured to not occlude any tubing line.

10. The device of claim 6, wherein when the dial is rotated to align with the third marker, the second occlusion surface is configured to occlude the patient line, and the first occlusion surface and the third occlusion surface are configured to not occlude any tubing line.

11. The device of claim 6, wherein when the dial is rotated to align with the fourth marker, the third occlusion surface is configured to occlude the drain line, and the first occlusion surface and the second occlusion surface are configured to not occlude any tubing line.

12. The device of claim 6, wherein when the dial is rotated to align with the fifth marker, the first occlusion surface is configured to occlude the patient line, the third occlusion surface is configured to occlude the drain line and the dialysis fluid line, and the second occlusion surface is configured to not occlude any tubing line.

13. The device of claim 2, wherein an angle between the drain line and the dialysis fluid line is smaller than either of an angle between the drain line and the patient line and an angle between the dialysis fluid line and the patient line when the at least one tubing line is engaged with the plurality of tubing pins.

14. The device of claim 2, wherein an angle between the drain line and the dialysis fluid line, an angle between the drain line and the patient line, and an angle between the dialysis fluid line and the patient line are all substantially equal when the at least one tubing line is engaged with the plurality of tubing pins.

15. The device of claim 3, wherein the dial further comprises a dial aperture configured to allow each of the plurality of markers to be viewed through the dial aperture when the dial is rotated to align with each of the plurality of markers.

16. The device of claim 1, wherein the base housing further comprises a locking pin configured to extend into a locking channel of the occluder plate when the CAPD device is in a closed configuration.

17. The device of claim 16, wherein the locking pin comprises an enlargement.

18. The device of claim 17, wherein the locking channel comprises at least one end aperture, wherein a diameter of the at least one end aperture is larger than a diameter of the enlargement.

19. The device of claim 17, wherein the locking channel comprises at least one lip configured to interfere with a bottom surface of the enlargement in at least one rotational position of the locking channel.

20. The device of claim 1, further comprising a latch configured to align with a base latch plate of the base housing and a top latch plate of the top housing,
wherein the latch comprises a latch through hole,
wherein the base latch plate comprises a base latch plate aperture,
wherein the top latch plate comprises a top latch plate aperture, and
wherein each of the latch through hole, the base latch plate aperture, and the top latch plate aperture are configured to align and accept a latch pin such that the latch couples the top housing to the base housing in a closed configuration of the CAPD device.
